(19) United States
(12) Patent Application Publication
(10) Pub. No.: US 2004/0267181 A1
(43) Pub. Date: Dec. 30, 2004

(54) Swab sample collection and recovery device

(75) Inventors: James Joseph Tuite, Gretna, VA (US); Peter Andreotti, San Francisco, CA (US)

Correspondence Address: BANNER & WITCOFF, 1001 G STREET N W, SUITE 1100, WASHINGTON, DC, 20001, US

(73) Assignee: ASD, Fort Lauderdale, FL

(21) Appl. No.: 10/866,135

(22) Filed: Jun. 14, 2004

Related U.S. Application Data
(60) Provisional application No. 60/482,517, filed on Jun. 26, 2003.

(51) Int. Cl. A61M 35/00; A61B 10/00

(52) U.S. Cl. 604/1

(57) ABSTRACT

A swab collection and recovery device comprising: an elongate tube having at least one side wall, an open top, and a closed bottom; a cap for closing the top of the tube; and a swab having a handle wherein one end of the swab comprises an absorbent material and the other end of the swab is attached to the inside of the cap, wherein the cap and the swab can be removed from the tube as a single unit; wherein the elongate tube further comprises a compressible indentation along the side wall, and the absorbent end of the swab can be squeezed at the position of the compressible indentation to express absorbed liquids into the bottom of the tube. A method of taking a culture sample comprising using the swab collection and recovery device.

This application claims priority to U.S. Provisional Application 60/482,517 filed Jun. 26, 2003, which is hereby incorporated by reference in its entirety.

### FIELD OF THE INVENTION

The present invention is directed to a swab sample collection and recovery device.

### BACKGROUND OF THE INVENTION

The ability to detect and identify biothreat and other disease causing agents in the environment requires the collection of samples for processing and analysis. This is commonly performed using a fiber tipped swab made of cotton, rayon, dacron, calcium alginate, or other material. These types of swabs suffer from different problems including nucleic acid contamination, which may interfere with genetic analysis of the sample, poor sample recovery due to the lack of efficient methods to elute analyte from the tip, and/or shredding of the tip during sample collection.

There are a number of important requirements for such a device. It must be applicable for spores, bacteria, viruses, proteins and other molecules, which may be potential biothreat or other disease causing agents. The device must also provide efficient collection and recovery of different agents from different types of samples. Efficient sample recovery must be feasible for portable field-testing with a minimum amount of laboratory equipment. The device must be compatible with different liquid solutions and methods used for analysis, and be inert and not shred so as not to interfere with analytical methods. The device must be an appropriate size for use by persons wearing protective clothing, and provide a means for safe transport and tracking of the sample.

U.S. Pat. No. 5,096,062 describes a complicated transport system to ship microbiological samples. The system comprises: (a) a first container provided with removable closure and containing a medium for storing the sample, and (b) a second container provided with a removable closure cap having an elongate chamber into which one end of a rod disposed therein can be pushed, said rod bearing a swab at its other end, the dimensions of the first and second containers, of the chamber in the closure cap and of the rod carrying the swab being so selected that the first container and a rod inserted therein in the medium and carrying the swab can be contained in the second container closed by the closure cap.

U.S. Pat. No. 4,586,604 discloses a culture collection instrument for use in animal husbandry including a tubular swab holder adapted to receive an elongated rod having a swab element on its end and a tubular swab protector adapted to receive and hold the tubular swab holder. The tubular swab protector includes a scored portion thereon a predetermined distance from the end of the swab protector such that flexing of the swab protector predeterminely breaks the swab protector and swab element from the elongated rod to provide a sealed tubular holder for the cultured swab element.

U.S. Pat. No. 4,789,639 describes a liquid recovery device including a receptacle, a swab for transferring a sample to a liquid in the receptacle, and a wringing unit to remove the liquid from the swab after the sample is transferred. The wringing unit has a sleeve with a closure in one end. A shaft of the swab extends through a passageway in the closure. When the swab in the receptacle is raised by upward movement of the shaft, an absorbent pad on the lower end of the shaft enters the passageway and is compressed. The compression of the pad wrings liquid therefrom which drips back into the receptacle. The device may be used in a method to prepare solutions of the sample in a minimum amount of liquid

U.S. Pat. No. 4,803,998 describes a device for obtaining samples from a patient's throat having a swab applicator, a containment vial and a cap for the vial. The swab applicator has a long shaft with a first section corresponding to the length of the vial and a second section joined to the first section by a frangible joint. After use of the applicator, the swab is inserted into the vial and the second section is broken off. A cap is placed on the vial whereby the cap captures the first section. The vial may be empty or contain a culture preserving substance.

U.S. Pat. No. 6,312,395 describes an apparatus for cell collection and transport having a tube and a polypropylene shaft having a cap attached at one end and a dental foam swab formed in the shape of a multi-apexed polygon affixed to the other end. Preferably at least 75% of the surface area of the cap is vented.

U.S. Pat. No. 5,916,802, U.S. Pat. No. 5,905,029 and U.S. Pat. No. 6,218,176 describe rapid hygiene testing systems that utilize a swab and tube.

A variety of swabs are known in the art such as those described by U.S. Pat. No. 6,393,651; U.S. Pat. No. 6,523,908; U.S. Pat. Nos. 5,084,005; and 6,004,640.

There also a variety of commercial swabs such as COPAN swabs (www.copanusa.com) which include swabs in tubes. The tubes have smooth round bottoms and one tube has an hour glass design for holding agar gel. There are also a number of BD BBL “Culture Swab” and “Culturette” products for swabs and transport systems (www.bd.com/clinical/products/collect). The tubes have an hour glass shape similar to the COPAN swabs.

There is a need for a better and more efficient device that can be used to collect and recover different types of samples from different types of environments and test the samples for a wide variety of agents with different methods. This is necessary for both portable field-testing and laboratory based testing.

### BRIEF SUMMARY OF THE INVENTION

The present invention is directed to a simple and easy-to-use sample collection and recovery device. The device utilizes a sterile swab with a foam tip that is packaged in a sterile, preferably conical-bottom, plastic storage and transport tube. The tube has a compressible indentation along the side wall of the tube. Preferably, the tube is disposable.

The swab handle is attached to the inside of a tube cap, which holds the swab inside the tube and provides a cap for the tube. The cap and swab assembly can be removed from the tube for sample collection. The cap and swab assembly can then be replaced for storage and transport of the swab in the tube, or for recovery of the sample from the swab into the tube. The swab may be detachable from the cap.

The conical tube bottom facilitates sample recovery from the swab tip by aiding the creation of a vortex of the buffer in the tube. The conical tube bottom also allows the eluted sample to be concentrated in the tube bottom by centrifugation. The compressible indentation along the side wall of the tube allows the liquid to be manually squeezed out of the swab tip for sample recovery.

The cap preferably has a gasket to form a tight seal with the tube. This allows a swab with a tip that is pre-moistened with a buffer solution to be packaged in the tube. This also allows a swab to be packaged in a tube containing a buffer solution, which may be used to moisten the swab tip and elute the recovered sample. In the case of samples collected from a biohazardous environment, the sealed tube also prevents specimen contamination and permits surface decontamination of the tube exterior for safe transport of the specimen without affecting the collected specimen.

The present invention is further directed to collection of environmental, human, veterinary, agricultural or industrial samples to detect and identify biothreat or other disease-producing agents. Each tube is coded with a unique identification medium, such as a unique barcode labeling or radio frequency identification device (RFID) implantation, to permit tracking of the specimen from the point of collection to the point of testing and/or archiving.

### BRIEF DESCRIPTION OF THE DRAWINGS

FIG. 1 shows one embodiment of a rectangular foam tip swab useful in the invention.

FIG. 2a shows a side view of one embodiment of the collection device tube

FIG. 2b shows another side view of the collection device tube shown in FIG. 2a.

FIG. 2c shows the tube opening at the top of the tube.

FIG. 3a shows a side view of one embodiment of the screw cap.

FIG. 3b shows an internal side view of the screw cap shown in FIG. 3a.

FIG. 3c shows a top view of the screw cap shown in FIG. 3a.

FIG. 3d shows an internal bottom view of the screw cap shown in FIG. 3a.

FIG. 3e shows a gasket for use in the cap.

### DETAILED DESCRIPTION OF THE INVENTION

The present invention is directed to a sample collection and recovery device. More specifically, the invention is directed to a manual device for sample collection and recovery that utilizes a swab and a storage and transport tube. The system is an improvement over prior art systems that require complicated combinations of tubes or caps. For instance, the liquid recovery device in U.S. Pat. No. 4,789,639 requires a wringing unit having two closures (46) and (42), placed through the top of a receptacle. The handle of the swab is pulled up through the cap of the receptacle forcing the absorbent tip through the closure (42) thus wringing out the liquid.

The present invention does not utilize an hour glass design such as described by COPAN tubes which are designed to hold gel and is not compressible. The COPAN design is not useful for expressing liquids as desired. The instant system allows insertion of a swab with a specimen into the liquid at the bottom of the tube, swirling of the liquid, and withdrawal of the swab whereby the swab is squeezed at the point of the compressible indent to express the liquid. Moreover, the COPAN tubes have a round bottom. Preferably, the tubes of the instant invention have a conical bottom to allow centrifuging.

The instant invention utilizes a tube with a compressible indentation. When a swab is removed from the tube, the swab is simply placed at the position of the compressible indent and the tube is squeezed. The swab can be removed from the cap and the cap replaced for storage and transport, or the cap can be replaced for storage and transport with the swab still attached to the cap, or the liquid may be removed at that point for testing. The entire system is simple and economical and does not require a wringing unit which adds cost to each recovery unit.

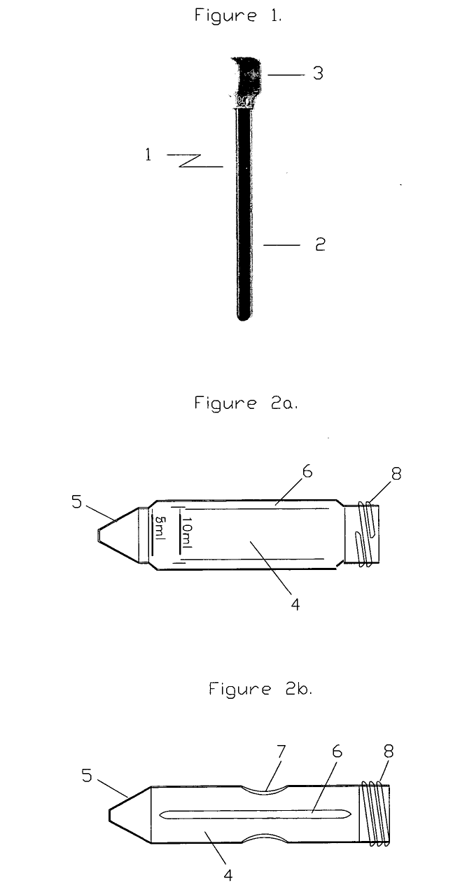

As shown in FIG. 1, the swab (1) has a handle (2) and a suitable tip (3). The tip is preferably made of porous foam material, more preferably open cell polyurethane. The foam tip is preferably a rectangular shape. The swab may be longer, wider, and thicker, but it is preferable to have a rectangular or square tip that is thin compared to its length and width. This geometric configuration for the swab tip improves sample collection and recovery compared to conventional ellipsoid cotton, rayon, Dacron, or other fiber swabs because a larger flat surface area is used to collect sample from a test surface, both sides of the tip can be used to collect a sample, and the thin porous foam tip allows efficient sample recovery by simply creating a vortex of the liquid within the tube or by compressing the tip. A desirable size for the swab tip is approximately 0.8-1.2 inches (20-30 mm) long×0.5-0.8 inches (12-20 mm) wide−0.3-0.5 inches (7-12 mm) thick.

The foam swab has additional advantages compared to conventional ellipsoid fiber swabs. The larger tip holds approximately 1 ml of sample liquid compared to approximately 0.2 ml for conventional fiber swabs. The foam tip also does not shred and is microbiologically inert. Shredding of the swab tip, which may occur with fiber tipped swabs, interferes with sample collection and analysis because of fiber contamination in the sample.

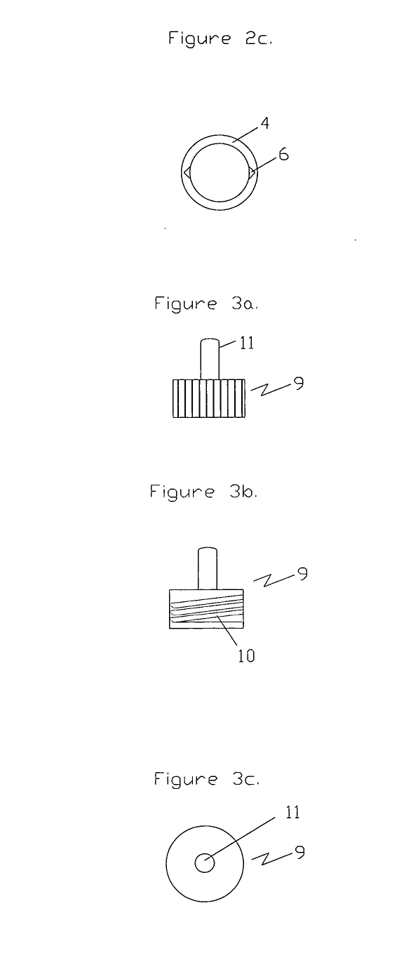

Attention is drawn to FIGS. 2a-2c. As shown in FIG. 2a, the storage and transport tube (4) has a bottom (5) and a spine (6) along the side wall which allows for compression of the tube at the compressible indent (7) shown in FIG. 2b. The indent optimizes recovery of the sample from the swab tip for detection and identification of biothreat or other disease producing agents. The compressible indentation is squeezable for expression of liquids from the swab tip. The tube should be sterile and is preferably plastic and disposable. A suitable material for the tube includes, but is not limited to, polypropylene. The optional threads (8) may be used to place and tighten the cap onto the tube. FIG. 2c shows the top of the tube (4) and position of spine (6).

The tube may have a round or conical bottom. The tube preferably has a conical bottom (5) as shown in FIG. 2a to facilitate the creation of a vortex with the buffer solution or any other liquid contained therein. The vortex may be created by motion of the hand or by using a suitable “vortex instrument” which may, for example, elliptically rotate the tube along its vertical axis. Creation of a vortex aids in the eluting of the, sample from the swab tip into the tube. The conical bottom also allows centrifuging to concentrate eluted sample in the bottom of the tube to optimize recovery. The tube has a compressible indentation along the side wall of the tube which allows the user to manually squeeze sample out of the foam tip while the swab tip is positioned inside the tube at the position of the compressible indentation.

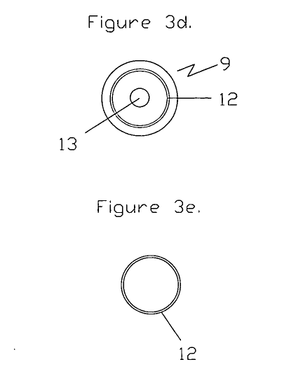

FIGS. 3a-3e show a cap (9) useful for placement on the tube. The cap is preferably a screw-type cap. FIG. 3a shows side view of the cap (a) showing cap handle (11). FIG. 3b shows an internal side view showing screw threads (10). FIG. 3c shows a top view of cap and position of the handle (11). FIG. 3d shows an internal bottom view of the cap with a gasket (12) around inside circumference of cap and the opening (13) for placement of a swab handle within the cap. The gasket ring or other type seal forms a tight seal with the tube to maintain the integrity of the sample and prevent loss of moisture or liquid from inside the tube during storage and transport. Suitable gaskets and seals are within the skill of the art. When it is sealed, the tube prevents specimen contamination and permits decontamination of the tube exterior for safe transport of the specimen without affecting the collected specimen

The cap preferably has a grippable surface to allow easy opening and tightening of the cap.

FIG. 3a shows a bottom view of the cap showing the slot (11) for the swab handle on the inside of the cap top. The end of the swab handle (2) is inserted into a slot (11) on the inside of the cap. The slot may be of any suitable design to hold the swab handle inside the tube. The swab handle can be pulled out of the slot to detach the swab from the cap so the swab can then be used independently from the cap. Alternatively, the swab handle can be permanently fixed in the slot. After the swab is detached from the cap and used independently, the swab handle can then be re-inserted into the slot to reattach the swab to the cap. The cap can then be screwed onto the tube. Alternatively, the cap may be used as an extended handle to obtain the sample.

The tube can contain a swab with a dry tip or a tip that is pre-moistened with a buffer solution for sample collection. A swab can be packaged in a tube that contains a buffer solution that may be used to moisten the swab tip for sample collection and elute sample from the swab tip into the tube. A useful amount of buffer solution is 3-10 ml.

The device can be used to collect and recover spores, bacteria, viruses, proteins and other molecules, which may be potential biothreat or disease causing agents. The device can be used to collect environmental, human, veterinary, agricultural, industrial or other types of samples from different environments.

Each storage and transport tube is preferably coded with a unique identification medium, such as a unique barcode labeling or radio frequency identification device (RFID) implantation to permit tracking of the specimen from the point of collection to the point of testing and/or archiving.

The device can be used in the field to collect a sample or collect and recover a sample. For instance, at the collection site, the cap is removed from the tube containing a swab. The cap can be used as a handle to manipulate the attached swab.

Collecting a sample with the swab requires the swab tip to be moistened. A dry swab may be moistened by adding a buffer solution directly to the test surface and then swabbing the surface with the swab. Alternatively, a swab with a tip that is pre-moistened with buffer solution can be packaged in a tube, or the tube may contain a buffer solution, for example 3-10 ml, which is used to pre-moisten the tip and elute the collected sample.

The test surface is swabbed with the tip of the swab to collect a sample. After the sample has been collected, the swab is replaced in the tube and the cap tightened to create a tight seal. The exterior of the sealed tube may be decontaminated by wiping or immersing it using bleach or other suitable solution to prevent cross contamination during storage and transport. The tube may be transported to a laboratory where the sample can be eluted from the foam tip for analysis. Alternatively, the device can be used to elute and recover the sample in the field for analysis.

To recover the sample in the field, the swab is replaced in the tube and the cap is tightened. The sample is then eluted and recovered in the tube. For example, a vortex is created with buffer solution contained in or added to the tube. Typically, 3-10 ml of buffer solution is adequate to achieve the elution. A standard buffer is Butterfield's Buffer or TRIS buffered saline (TBS) containing a detergent such as 0.02%-0.01% Tween-20 or 1.0%-2.0% pluronic acid. The sample can also be eluted from the swab tip by manually compressing the swab tip inside the tube when the tip is positioned at the compressible indentation along the side wall of the tube. This manual elution of the sample using the compressible indentation can be performed with or without additional buffer solution added to the tube. Alternatively, both creation of a vortex and manual compression of the swab tip can be used together to obtain sample elution. Particulate sample recovery can be further optimized by centrifugation of the eluted sample to concentrate sample at the conical bottom of the tube.

After the sample has been eluted and recovered for analysis, the tube can be sealed with the cap for transport to a laboratory for additional analysis. The device can also be used for sample collection and analysis without first eluting the sample from the swab tip by using the foam swab to streak out the sample onto a petri dish containing growth medium, or inserting the swab tip into liquid growth medium to inoculate a culture.

The results in Table 1 demonstrate the greater absorption and recovery of spores, bacteria and protein obtainable with the foam swab compared to conventional fiber swabs. Foam swabs were compared with conventional cotton, rayon, Dacron and calcium alginate swabs using 10 swabs of each type for each experiment. The swabs were first compared for the volume of liquid that could be absorbed into the tip using aliquots of Butterfields Buffer pH 7.2 added to the tip with a micropipet. Then the swabs were compared for the volume of liquid that could be eluted from the tip using a micropipet to measure the eluted buffer after manually compressing the tip. As can be seen in Table 1, the foam swabs can absorb 4.20-5.25 times the volume of sample liquid compared to conventional fiber swabs, and elute up to 93% of the absorbed liquid with simple manual manipulation compared to 88% or less for the fiber swabs. This increase in absorption capacity and elution efficiency makes it possible to collect and recover a sample volume that is up to 5.5 times greater than what can be obtained with conventional fiber swabs.

Swabs were also compared for absorption and elution of Bacillus globisporus (BG) spores, vegetative Salmonella typhimurium, and protein (toxin simulant) using firefly luciferase enzyme. 0.5 ml of water containing approximately 31,000 colony forming units (CFU) of BG spores, 0.5 ml of saline containing approximately 27,000 CFU of Salmonella typhimurium, and 10 ng of luciferase in 0.25 ml of water containing 0.1% bovine serum albumin were dried in spots of approximately 0.05 ml each on the bottom of sterile 150 mm polystyrene petri dishes.

Foam and fiber swab tips were then saturated with Butterfields Buffer containing 2.0% pluronic acid detergent and used to swab the bottom surface area of the petri dishes to collect the dried BG spore, Salmonella typhimurium, and luciferase samples. Fiber swabs were then eluted into 9.8 ml of buffer while foam swab were eluted into 9.1 ml of buffer in 50 ml conical centrifuge tubes by vortexing the tube containing the swab in the buffer and then manually compressing the swab tip along the inside wall of the tube.

Butterfields buffer containing 2.0% pluronic acid was used for elution of BG spore and Salmonella typhimurium samples while luciferase samples were eluted into 0.1 M Hepes buffer pH 7.9. Eluted BG spore and Salmonella typhimurium samples were diluted 1:10 and 1:100 in elution buffer, 0.3 ml aliquots of each dilution were then plated on nutrient agar plates, and CFU were counted after 24 hours incubation at 37° C. Percent recovery was calculated from 0.5 ml control BG spore or Salmonella typhimurium samples that were added to 10 ml of elution buffer and then diluted 1:10 and 1:100 before plating on nutrient agar. 0.05 ml of each luciferase eluate was added to 0.1 ml of Hepes Buffer pH 7.9 containing 0.25 mg/ml D-luciferin and 100 ng of ATP and the counts per second (cps) for light output of the mixture was measured in a luminometer. Percent recovery was calculated from 0.5 ml control luciferase (10 ng) samples that were added to 10 ml of Hepes buffer and then measured in the luminometer after 0.05 ml to 0.1 ml of buffer containing D-luciferin and ATP. Under these conditions with excess D-luciferin and ATP, the light output is linearly proportional to the concentration of recovered luciferase. As can be seen in Table 1, the recovery efficiency for spores, bacteria and protein was consistently greater than 91% for the foam swabs compared to 6.3%-83.0% for the conventional fiber swabs.

TABLE 1
Absorption And Recovery Of Foam Swab Compared
to Conventional Fiber Swabs

| | Poly- | | | | |
|---|---|---|---|---|---|
| | urethane | Cotton | Rayon | Dacron | Calcium |
| | Foam | Fiber | Fiber | Fiber | Alginate |
| Volume Of Liquid | 1.05 ml | 0.23 ml | 0.25 ml | 0.25 ml | 0.2 ml |
| Absorbed | | | | | |
| Volume Of Liquid | 0.98 ml | 0.17 ml | 0.22 ml | 0.22 ml | 0.15 ml |
| Eluted | | | | | |
| Mean Total BG | 28,646 | 21,873 | 25,295 | 23,775 | 18,348 |
| Spore CFU | (91.2%) | (69.6%) | (80.5%) | (75.7%) | (58.4%) |
| Recovered¹ | | | | | |
| Mean Total | 25,221 | 21,889 | 22,587 | 20,252 | 12,483 |
| Salmonella CFU | (92.7%) | (80.4%) | (83.0%) | (74.4%) | (45.9%) |
| Recovered² | | | | | |
| Mean Luciferase cps | 387,723 | 113,722 | 187,859 | 157,982 | 25,745 |
| Recovered³ | (94.8%) | (27.8%) | (45.9%) | (38.6%) | (6.3%) |

¹Values in parenthesis show calculated percent recovery based on an input of 31,408 CFU determined by plating of controls.
²Values in parenthesis show calculated percent recovery based on an input of 27,211 CFU determined by plating of controls.
³Values in parenthesis show calculated percent recovery based on an input of 408,933 cps determined by counting of controls.

The device can be used by simple creation of manual compression of the swab tip, or a combination of a liquid vortex and manual compression of the tip to achieve efficient sample recovery.

Table 2 shows results obtained with different elution methods using 10 swabs for each method that were absorbed with approximately 1775 CFU of BG spores in 0.75 ml of Butterfields Buffer pH 7.2 containing 2.0% pluronic acid detergent. After absorbing the BG spores into each swab tip, elution was performed by immersing the swab tip in a vortex of 9.25 ml of buffer for 15 seconds, dipping and then manually compressing the tip in 9.25 ml of buffer, or both immersing the swab tip in a vortex and manually compressing the tip in 9.25 ml of buffer.

After elution, 0.3 ml aliquots of each eluate were plated onto nutrient agar and CFU were counted after 24 hours incubation at 37° C. Percent recovery was calculated from 0.75 ml control samples that were directly added to 9.25 ml of buffer and then plated on nutrient agar. As can be seen in Table 2, the recovery efficiency for each method was high and at least 95.2%.

TABLE 2
Recovery Of BG Spores From Foam
Swab Using Different Elution Methods

| | Mean Total | Standard | | Percent |
|---|---|---|---|---|
| | CFU | Deviation | CV % | Recovery |
| Control Sample Not | 1775 | 220 | 12.4% | 100% |
| Absorbed & Eluted | | | | |
| Vortex Eluted Sample | 1689 | 320 | 18.9% | 95.2% |
| Manual Compression | 1717 | 189 | 11.0% | 96.7% |
| Eluted Sample | | | | |
| Vortex & Manual | 1804 | 112 | 6.2% | 102% |
| Compression | | | | |
| Eluted Sample | | | | |

The device utilizes a unique storage and transport tube that is also designed to optimize sample recovery from the swab. The tube is preferably disposable and made of plastic.

An important consideration for sample collection and recovery is the ability of the user to hold and manipulate the device when wearing protective clothing, which may involve simultaneously wearing 2-3 pairs of gloves. The tube is approximately 4.5-6.0 inches (114-150 mm) long with a diameter of approximately 1.0-1.5 inches (25-38 mm) and an approximately 0.8-1 inch (20-25 mm) conical bottom. The tube may be larger or smaller, but this size range allows the user to easily hold and manipulate the device with gloved hands. This size range is also preferable for the tube to fit established laboratory apparatus including test tube racks, vortex mixers, and centrifuges which are widely used for 50 ml conical centrifuge tubes. The conical tube bottom facilitates creation of a vortex within the tube to elute sample from the swab tip when it is immersed in buffer in the tube. The conical tube bottom also facilitates concentrating particulate analytes, such as spores and bacteria, by centrifuging the tube containing eluted sample.

The tube has sufficient rigidity to centrifuge eluted sample in the tube, yet it also has a compressible indentation along the side walls preferably approximately 2.0-3.9 inches from the bottom of the tube. The compressible indentation is designed to manually compress the swab tip to elute sample from the tip into the tube. The compressible indentation may be any suitable length, but preferably is approximately 0.7-0.9 inches long, and allows the user to completely compress the swab tip between the thumb and forefinger while the swab tip is positioned inside the tube between the indentation.

The tube is sealed, preferably with a screw-on cap and gasket, to prevent cross contamination of the sample and evaporation of liquid during storage and transport of the device. The swab handle is reversible attached to the inside of the tube cap which holds the swab inside the tube and serves as a handle to manipulate the swab after the cap is remove from the tube. The swab can be removed from the handle for additional sample collection, recovery, and processing procedures in the field or laboratory. The swab can be permanently fixed to the cap which can be used as handle to manipulate the attached swab.

A typical method for sample collection without sample elution and recovery in the field utilizes the following procedure with a tube containing a pre-moistened swab:

1. Unscrew the tube cap and remove the cap and pre-moistened swab assembly from tube.

2. Swab a test area of approximately 11″×11″ using the tube cap to manipulate the swab.

3. Insert the swab back into the tube and seal the tube with the screw cap for storage and transport.

A typical method for sample collection, elution and recovery in the field or laboratory utilizes the following procedure with a tube containing 3-10 ml of elution buffer solution:

1. Unscrew the tube cap and remove the pre-moistened swab from tube.

2. Swab a test area of approximately 11″×11″ using the tube cap to handle the swab.

3. Insert the swab back into the tube and seal the tube with the screw cap.

4. Create a vortex of the solution in the tube to elute sample in the swab tip that is immersed in the buffer solution.

5. Loosen tube cap and position the swab tip inside the tube at the compressible indentation.

6. Squeeze the swab tip between the plastic side walls of the tube at the compressible indentation to elute sample from the tip into the buffer solution in the tube.

7. Remove sample aliquot from the tube for analysis.

8. Re-seal the tube with the screw cap for storage and transport.

A typical method for collection, elution, concentration and recovery of a particulate sample in the field or laboratory utilizes the following procedure with a tube containing 3-10 ml of buffer solution:

1. Unscrew the tube cap and remove the pre-moistened swab from tube.

2. Swab a test area of approximately 11″×11″ using the tube cap to handle the swab.

3. Insert the swab back into the tube and seal the tube with the screw cap.

4. Create a vortex with the liquid in the tube to elute sample in the swab tip that is immersed in the buffer solution.

5. Unscrew tube cap and position the swab tip inside the tube at the compressible indentation.

6. Squeeze the swab tip between the plastic side walls of the tube at the compressible indentation to elute sample from the tip into the buffer solution in the tube.

7. Re-seal the tube with the screw cap.

8. Centrifuge the tube to pellet the particulate sample at the bottom of the tube.

9. Unscrew tube cap and remove excess buffer solution with a pipet.

10. Resuspend concentrated sample in remaining 0.5-1.0 ml of buffer solution.

11. Remove sample aliquot from the tube for analysis.

12. Re-seal the tube with the screw cap for storage and transport.

While the invention has been described with respect to specific examples including presently preferred modes of carrying out the invention, those skilled in the art will appreciate that there are numerous variations and permutations of the above described systems and techniques that fall within the spirit and scope of the invention.

1. A swab collection and recovery device comprising:
an elongate tube having at least one side wall, an open top, and a closed bottom;
a cap for closing the top of the tube; and
a swab having a handle wherein one end of the swab comprises an absorbent material and the other end of the swab is attached to the inside of the cap, wherein the cap and the swab can be removed from the tube as a single unit;
wherein the tube further comprises a compressible indentation along the side wall, and the absorbent end of the swab can be squeezed at the position of the compressible indentation to express absorbed liquids into the bottom of the tube.

2. The device according to claim 1 wherein the handle is detachable from the cap.

3. The device according to claim 1 wherein the handle is not detachable from the cap.

4. The device according to claim 1 wherein the bottom of the tube is conical.

5. The device according to claim 1 wherein the cap is a screw cap and the top of the tube has threads to receive the cap.

6. The device according to claim 1 wherein the cap comprises a gasket to form a seal with the tube.

7. The device according to claim 1 wherein the absorbent material is foam.

8. The device according to claim 7 wherein the foam is polyurethane.

9. The device according to claim 7 wherein the foam tip is rectangular.

10. The device according to claim 1 further comprising an identification medium attached to the tube to permit tracking of the specimen from the point of collection to the point of testing or archiving.

11. The device according to claim 10 wherein the identification medium is a barcode or a radio frequency identification device implantation.

12. A method of taking a culture sample comprising:
applying a swab having an absorbent end pre-moistened with a liquid to a surface to collect a sample;
inserting the swab into an elongate tube, wherein the tube has at least one side wall, an open top, a closed bottom, and a compressible indentation along the side wall, wherein the tube contains liquid at the bottom;
immersing the absorbent end of the swab in the liquid;
positioning the swab at the position of the compressible indentation;
squeezing the tube thereby squeezing the absorbent end of the swab to express the absorbed liquid into the bottom of the tube.

13. The method of claim 12 further comprising removing the swab from the tube prior to applying the swab to the surface.

14. The method of claim 12 wherein the swab has a handle attached to the inside of a cap and using the cap to manipulate the swab and to seal the tube.

15. The method of claim 14 further comprising attaching the cap to the top of the tube so as to effect a sealing engagement between said cap and the tube.

16. The method of claim 12 further comprising, after inserting the absorbent end of the swab into the liquid at the bottom of the tube, creating a vortex with the liquid in the bottom of tube.

17. The method of claim 12 further comprising, after squeezing the tube, centrifuging the tube.

18. The method of claim 12 further comprising, after squeezing the tube, removing the swab from the tube.

19 The method of claim 14 further comprising, after squeezing the tube, removing the swab from the tube and the cap, and replacing the cap on the tube.
